(19) United States
(12) Patent Application Publication
(10) Pub. No.: US 2022/0096365 A1
(43) Pub. Date: Mar. 31, 2022

(54) NATURAL SKIN CARE SOLUTION

(71) Applicant: Marcia Peeples, New Rochelle, NY (US)

(72) Inventor: Marcia Peeples, New Rochelle, NY (US)

(21) Appl. No.: 17/487,331

(22) Filed: Sep. 28, 2021

Related U.S. Application Data
(60) Provisional application No. 63/084,119, filed on Sep. 28, 2020.

(51) Int. Cl. A61K 8/9789; A61K 8/19; A61K 8/92; A61K 8/67; A61Q 19/00

(52) U.S. Cl. CPC A61K 8/9789; A61K 8/19; A61K 8/922; A61K 2800/10; A61K 8/678; A61K 8/927; A61Q 19/00; A61K 8/925

(57) ABSTRACT

Compositions for body, skin, and haircare are disclosed herein. In a non-limiting embodiment, a method for making a composition may include combining baking soda with arrowroot powder to create a dry mixture, and adding coconut oil to the dry mixture. The method may further include combining the dry mixture with at least one of emu oil, jojoba oil, and vitamin E, and then combining the heated coconut oil and the dry mixture for a predetermined amount of time to create a viscous mixture. The method may also include heating an amount of beeswax to a predetermined temperature, emulsifying the viscous mixture in the beeswax, and then reducing the temperature of the beeswax to room temperature to create a soft, yet substantially solid composition.

### FIELD OF THE INVENTION

This invention relates to lotions, balms, and other compositions to be applied to skin, hair, and other areas of a body. For instance, exemplary embodiments of this invention may be applied to human skin to moisturize the skin, as well as protect it from external hazards such as sunlight, cold weather, and dry weather.

### DETAILED DESCRIPTION

A first object of the invention is to create a formulation that contains natural ingredients, including but not limited to: baking soda; coconut oil, arrowroot powder; emu oil; jojoba oil, vitamin E oil; and beeswax.

In a preferred embodiment, these natural ingredients may be organic. Additionally, these ingredients may be incorporated into a composition in various proportions based on a quality of a person's skin, hair, or other bodily organ upon which the inventive composition is to be applied.

Baking soda includes various skin care benefits. For instance, baking soda may act as an exfoliant and antimicrobial agent to promote softer, cleaner skin. Coconut oil may act as an anti-inflammatory and antimicrobial agent to protect skin from microbial growth, acne, and other hazards to the skin. Arrowroot may serve as a natural thickener, opacifier, and softener that can absorb moister, assist in masking body odors, and provide increased structural integrity to various compositions in accordance with the invention. Emu oil may act as an anti-inflammatory and emollient, and can assist in moisturizing skin and reducing damage caused by ultraviolet radiation from the sun. Jojoba coil may also act as an anti-inflammatory, and can also include various nutrients that can reduce skin irritation. Vitamin E is a fat soluble vitamin that may act as an antioxidant and may be effective at reducing damage caused by ultraviolet radiation from the sun. Beeswax may also be anti-inflammatory and provide structural integrity to compositions in accordance with the invention.

In an embodiment, a formulation may be combined according to the following method. First, dry ingredients may be combined. For instance, baking soda and arrowroot powder may be combined using any means known in the art. For example, baking soda and arrowroot powder may be combined in a mortar and pestle, a food processor, an immersion blender, or some other means for combining dry ingredients. Second, emu oil, jojoba oil, and vitamin E oil are added to the dry mixture. These oils may be added separately, one by one, or may alternatively be combined into a separate emu/jojoba/vitamin E oil, and then added as a combined oil mixture to the dry mixture. Third, coconut oil may be added to the mixture. In an embodiment, the coconut oil may be heated to a predetermined temperature (at least 78 degrees Fahrenheit) in order to ensure the coconut oil is in a liquid state. Similarly to the first step, these ingredients may be combined using an immersion blender, a food processor or some other means for combining ingredients.

At a fourth step, beeswax is heated to a predetermined temperature (e.g., approx. between 140 and 400 degrees Fahrenheit), until the beeswax is in a liquid state, which may occur over a course of roughly 3 to 8 minutes. The heating of the beeswax may occur while ingredients are being combined in the first through third steps, or may occur before or after. At step five, once the ingredients are substantially mixed together and the beeswax has reached the predetermined temperature and liquid state, the beeswax is added to the mixture until the mixture is substantially mixed into the beeswax, the combined mixture is left to cool to room temperature, at which time it will be ready for use.

In one embodiment of the invention, ingredients may be mixed in such a manner to provide maximum reactivity with human skin. For instance, an exemplary composition may be composed according to the following proportions:

| | Amount (in U.S. | | | | By Percent |
|---|---|---|---|---|---|
| Ingredient | customary units) | | Amount (in parts) | | (approx.) |
| Baking Soda | 2 | tablespoons | 24 | parts | 11.5%-15.0% |
| Coconut oil | 3 | tablespoons | 36 | parts | 17.3%-22.5% |
| Arrowroot | 2 | tablespoons | 24 | parts | 11.5%-15.0% |
| powder | | | | | |
| Emu oil | 0.25 | teaspoon | 1 | part | 0.5%-0.6% |
| Jojoba oil | 0.5 | teaspoon | 2 | parts | 1.0%-1.2% |
| Vitamin E oil | 0.25 | teaspoon | 1 | part | 0.5%-0.6% |
| Beeswax | 3 to 5 | ounces | 72 to 120 | parts | 17.0%-57.7% |

The variation in the amount of beeswax may depend on a variety of factors. In an embodiment, the beeswax constitutes roughly 1.5 to 2.5 ounces, or 36 to 60 parts in proportion to other components of the composition. In another embodiment, another composition may be provided for sensitive skin. For instance, a combination may be composed in accordance with the following proportions.

| | Amount (in U.S. | | | | By Percent |
|---|---|---|---|---|---|
| Ingredient | customary units) | | Amount (in parts) | | (approx.) |
| Baking Soda | 1 | tablespoons | 12 | parts | 6.1%-8.1% |
| Coconut oil | 3 | tablespoons | 36 | parts | 18.3%-24.3% |
| Arrowroot | 2 | tablespoons | 24 | parts | 12.2%-16.2% |
| powder | | | | | |
| Emu oil | 0.25 | teaspoon | 1 | part | 0.5%-0.7% |
| Jojoba oil | 0.5 | teaspoon | 2 | parts | 1.0%-1.4% |
| Vitamin E oil | 0.25 | teaspoon | 1 | part | 0.5%-0.7% |
| Beeswax | 3 to 5 | ounces | 72 to 120 | parts | 48.6%-61.2% |

Baking soda may be a cause of irritation to the skin due to its coarse nature, or to potential allergens. Arrowroot powder may assist in neutralizing this possible side effect. Therefore, it may be beneficial to adjust the ratio of baking soda to arrowroot powder such that a larger proportion of arrowroot powder is included in the composition in comparison with the baking soda. Accordingly, a reduction in the amount of baking soda in the final composition may be helpful to provide a composition more comfortable for sensitive skin.

Persons of ordinary skill in the art will appreciate that the embodiments discussed herein are only disclosed for the purpose of illustrating the structure, function, and benefits (among others) of the inventions disclosed herein. As such, persons of ordinary skill in the art will appreciate that these embodiments are not limiting, and that the inventions disclosed here in include other embodiments not expressly disclosed herein.

What is claimed is:

1. A composition, comprising:
baking soda;
coconut oil;
arrowroot powder;
emu oil;
jojoba oil;
vitamin E;
yellow beeswax.

2. The composition of claim 1, wherein the composition comprises at least 11 percent baking soda, at least 17 percent coconut oil, at least 11 percent arrowroot powder, at least 0.5 percent emu oil, at least 1 percent jojoba oil, at least 0.5 percent vitamin E oil, and at least 17 percent yellow beeswax.

3. A method for making a composition, comprising:
combining baking soda with arrowroot powder to create a dry mixture;
adding coconut oil to the dry mixture;
combining the dry mixture with at least one of emu oil, jojoba oil, and vitamin E;
combining the heated coconut oil and the dry mixture for a predetermined amount of time to create a viscous mixture;
heating an amount of beeswax to a predetermined temperature;
emulsifying the viscous mixture in the beeswax; and
reducing the temperature of the beeswax to a predetermined temperature.

4. The method of claim 3, further comprising heating the coconut oil to at least 78 degrees Fahrenheit prior to adding the coconut oil to the dry mixture.

5. The method of claim 3, wherein heating the amount of beeswax comprises heating the beeswax to at least 140 degrees Fahrenheit.

6. The method of claim 3, wherein heating the amount of beeswax comprises heating the beeswax to at least 400 degrees Fahrenheit.

7. The method of claim 6, wherein heating the amount of beeswax occurs over a temporal interval between three minutes and eight minutes.
